(19) United States
(12) Patent Application Publication
(10) Pub. No.: US 2008/0154140 A1
(43) Pub. Date: Jun. 26, 2008

(54) Carotid pulse measurement device

(76) Inventors: Chien-Cheng Chang, Taipei City (TW); Po-Hsiang Tsui, Jhonghe City (TW); Chien-Chung Chang, Taipei City (TW); King-Jen Chang, Taipei City (TW); Chung-Li Wang, Taipei City (TW); Juey-Jen Hwang, Taipei City (TW); Chin-Chou Chu, Taipei City (TW); Chiung-Nien Chen, Taipei City (TW); Lian-Yu Lin, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/783,447

(22) Filed: Apr. 10, 2007

(30) Foreign Application Priority Data
Dec. 22, 2006 (TW) 95148383

(51) Int. Cl. A61B 5/02

(52) U.S. Cl. 600/500

(57) ABSTRACT

A carotid pulse measurement device provided for measuring carotid pulse of a user's neck includes a band, an artery-pressing air cell, an inflating/deflating device, and a pressure detection unit. The band functions to surround the user's neck. The air cell is arranged in the band and is fluidly connected to the inflating/deflating device for selectively inflating/deflating the air cell. The pressure detection unit detects variation of pressure inside the air cell. To use the measurement device, the band is put around the user's neck with the air cell positioned exactly corresponding to the carotid artery of the neck so that the pressure detection device detects the pressure variation and generates a digital pulse signal, which is employed to calculate arteriosclerosis index of the user for evaluation the degree of arteriosclerosis of the user.

### FIELD OF THE INVENTION

The present invention relates to a pulse measuring device, and in particular to a carotid pulse measurement device.

### BACKGROUND OF THE INVENTION

Factors including intima media thickness (IMT), pulse wave velocity (PWV), and pulse waveform are commonly used as an arteriosclerosis index by doctors to evaluate the degree of arteriosclerosis of a patient. The degree of arteriosclerosis is in turned used to predict the occurrence of strokes or other vessel diseases.

Conventionally, IMT is measured with ultrasonic based instruments or non-invasion blood vessel inspection methods. An example is disclosed in Taiwan Earlier Publication No. 200500041, which employs both ultrasonic instrument and pressure detection unit to simultaneously monitor the blood pressure and diameter of blood vessel. Appropriate ultrasonic fixation device and special imaging technology are taken to obtain the mechanical property of the peripheral artery vessel and to measure IMT.

In addition, Taiwan Earlier Publication No. 200425873 teaches measuring time lag between waveforms of blood flow pulse at different arteries to indicate the degree of expansion of blood vessel and eventually detecting the effectiveness of regulation performed by the endothelial cell, which can serve as a basis for evaluating degree of arteriosclerosis. Taiwan Patent No. 514514 discloses a device for evaluating the degree of arteriosclerosis in vivo by measuring the pulse at a portion of the body and evaluating arteriosclerosis based on a predetermined relationship among data related to pulse speed, data related to increase of amplitude of the pulse, and the degree of arteriosclerosis. Taiwan Patent No. 546129 discloses an arteriosclerosis evaluating apparatus for evaluating arteriosclerosis in vivo, which detects the pulse waves at a first portion and a second portion of the subject body. The arteriosclerosis evaluating apparatus comprises an augmentation-index determining means for determining the augmentation index indicative of a degree of augmentation of an amplitude of the pulse wave detected by the pulse-wave detecting device. An arteriosclerosis evaluating means evaluates the arteriosclerosis of the body by comparing the first and second augmentation indexes of the increase of the amplitude of the pulse wave of the first portion and that of the second portion.

Taiwan Patent Publication No. 534807 discloses an augmentation-index determining apparatus and arteriosclerosis inspecting apparatus, which uses a cuff-pressure changing device to vary the pressure of the cuff. The time of occurrence of a peak point of an incident-wave component of the high-cuff-pressure pulse and a time of occurrence of a peak point of a reflected-wave component, and that for a low-cuff-pressure pulse are determined. From the data, the augmentation indexes are determined.

To summarize, the conventional techniques for IMT measurement are done with ultrasonic instruments. The conventional techniques, however, are complicated processes, which cannot be performed by an individual. In addition, the device itself is bulky and is not suitable for home use. Further, IMT itself does not always correctly reflect the degree of arteriosclerosis.

Further, other measurement devices are operated to measure the blood flows and blood pressures in arms, fingers, and toes for calculating the transmission speed of blood flow and blood pressure in arteries or related parameters of the pulse waveform, or for estimating IMT value to serve as an index for accessing arteriosclerosis. However, to include more information of heart and brain blood vessels in the arteriosclerosis index, the pulse measurement device must be positioned close to the bloods vessels of heart and brain, rather than peripheral vessels, and using the pulse signal of the blood vessels to calculate the arteriosclerosis index. In this respect, carotid artery is the vessel that is closest to the heart and brain and good for non-invasion measurement. Thus, blood vessel pulse signal obtained from the carotid artery can be used to determine a carotid arteriosclerosis index that can predict the risk of vessel diseases and stroke in a more precise manner.

Currently, the most commonly seen device for measuring pulse comprises an inflatable cuff. The known inflatable cuff is wholly inflated to carry out measurement. This often causes discomfort due to pressure applied to the neck.

The present invention is aimed to overcome the drawbacks of the conventional devices.

### SUMMARY OF THE INVENTION

Thus, an objective of the present invention is to provide a carotid pulse measurement device comprising a pressure detection unit that detects the pressure variation of an air cell to measure the pulse of the carotid artery.

Another objective of the present invention is to provide an artery-pressing air cell that comprises a plurality of inflation chambers wherein the chambers are arranged in a stacked manner and are in fluid communication with each other so that the pressure detection unit can do more sensitive measurement of the pressure variation of the air cell.

A further objective of the present invention is to provide an artery-pressing band comprising locally arranged effective artery-pressing section to measure the carotid pulse instead of inflating the whole band as done in the conventional devices.

Yet a further objective of the present invention is to provide an arteriosclerosis evaluation device, which uses a pressure detection unit to detect pressure variation of an artery-pressing air cell and also uses a pulse processing module to evaluate the degree of arteriosclerosis.

To realize the above objectives, the present invention provides a carotid pulse measurement device or measuring carotid pulse of a user's neck, comprising a band, an artery-pressing air cell, an inflating/deflating device, and a pressure detection unit, wherein the band comprises an enclosing section and an effective pressing section. The air cell is arranged in the band at a location corresponding to the effective pressing section. The air cell is fluidly connected to the inflating/deflating device via an inflation tube for selectively inflating/deflating the air cell. The pressure detection unit is also fluidly connected to the inflation tube of the inflating/deflating device to detect variation of pressure inside the air cell.

When the inflating/deflating device is operated to inflate the air cell, the pressure detection unit detects the pressure variation of the inflation tube of the inflating/deflating device and the air cell and generates a pulse signal that is applied to and processed by a pulse signal processing module to convert the pulse signal into a digital pulse signal.

In another embodiment of the present invention, the pressure detection unit may be arranged between an air-contact surface of the band and the air cell to allow the pressure detection unit to detect the pressure variation of the air cell by means of a reaction induced by the inflated air cell with respect to the carotid pulse.

Further, in a preferred embodiment of the present invention, the measurement device may comprise a first air cell and a second air cell arranged in the band at locations corresponding to left and right carotid arteries. The second air cell is fluidly connected to the first air cell by an inter-cell inflation tube, or alternatively, the second air cell is directly and fluidly connected to the inflating/deflating device. The first and second air cells may generate individual pulse signals, which can provide the pulse signal processing module with pulse signals of the left and right carotid arteries and also allows comparison and combination of the two pulsing conditions or to combine with pulse waves of peripheral vessels or extremity vessels for calculation of arteriosclerosis index.

As compared to the existing techniques, the present invention allows for measuring carotid pulse in a non-invasive manner in order to calculate arteriosclerosis index. In addition, the first and second pressure detection units that are used in the present invention allow for simultaneously measuring the pulse signals of the left and right carotid arteries. This provides complete information regarding carotid pulsing and also reduces error caused by improperly wearing of the device on the user's neck. Thus, the present invention features both precise measurement of carotid pulse and easy operation by an individual, and is thus suitable for home use. In addition, the present invention employs local pressing by means of the air cell arranged in the effective pressing section whereby strain and discomfort caused on the user's neck by the band surrounding the neck are alleviated as compared to the conventional devices.

### BRIEF DESCRIPTION OF THE DRAWINGS

The present invention will be apparent to those skilled in the art by reading the following description of preferred embodiments thereof, with reference to the attached drawings, in which:

FIG. 1 is a perspective view showing a carotid pulse measurement device constructed in accordance with a first embodiment of the present invention used to measure the carotid pulse of a user;

FIG. 2 is a perspective view of a flexible band of the carotid pulse measurement device of the first embodiment of the present invention;

FIG. 3 is a cross-sectional view taken along line 3-3 of FIG. 2;

FIG. 4 is a system block diagram of the carotid pulse measurement device of the first embodiment of the present invention;

FIG. 5 is a perspective view showing a carotid pulse measurement device constructed in accordance with a second embodiment of the present invention used to measure the carotid pulse of a user;

FIG. 6 is a perspective view of a flexible band of the carotid pulse measurement device of the second embodiment of the present invention;

FIG. 7 is a cross-sectional view taken along line 7-7 of FIG. 6;

FIG. 8 is a system block diagram of the carotid pulse measurement device of the second embodiment of the present invention; and

FIG. 9 is a perspective view of a flexible band of a carotid pulse measurement device constructed in accordance with a third embodiment of the present invention.

### DETAILED DESCRIPTION OF THE PREFERRED EMBODIMENTS

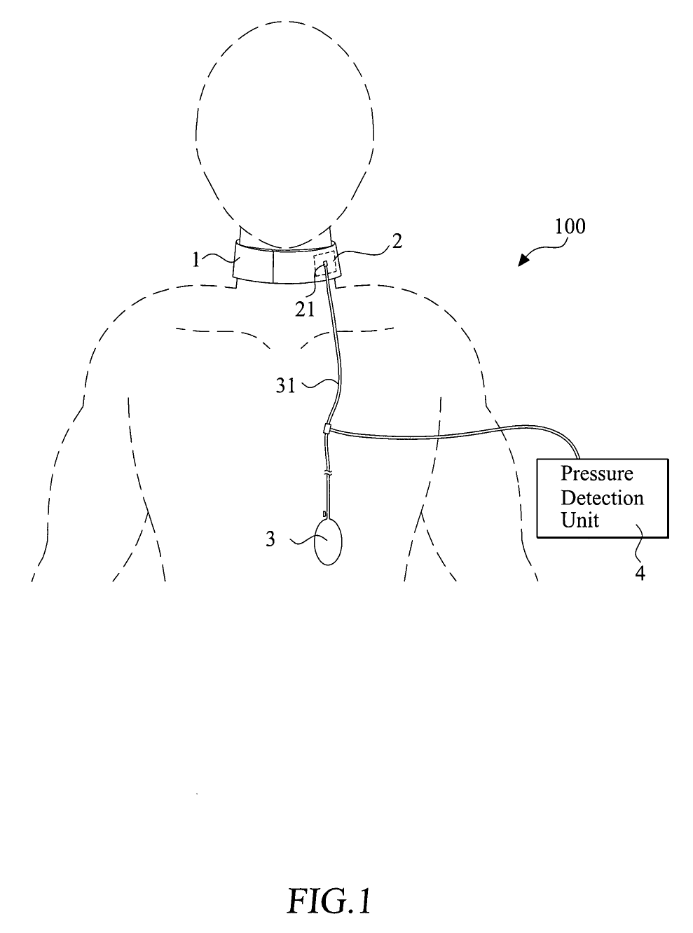

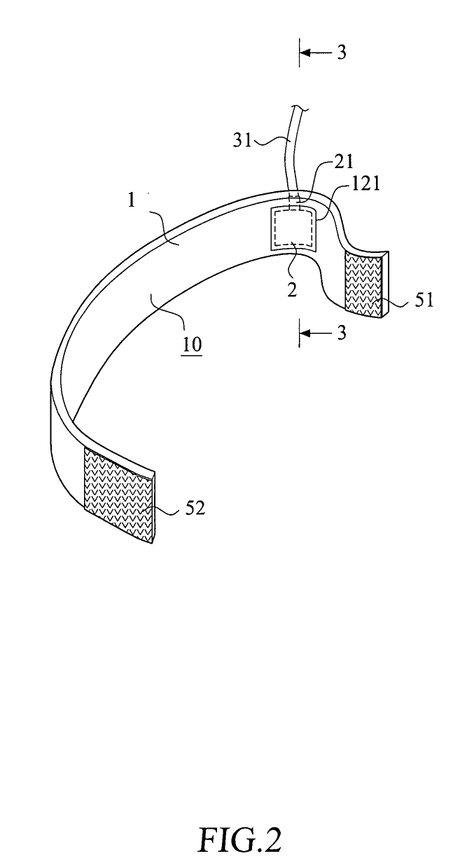

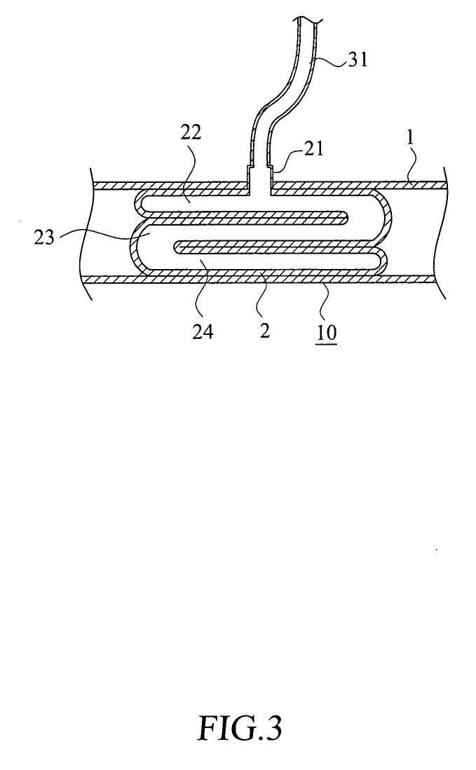

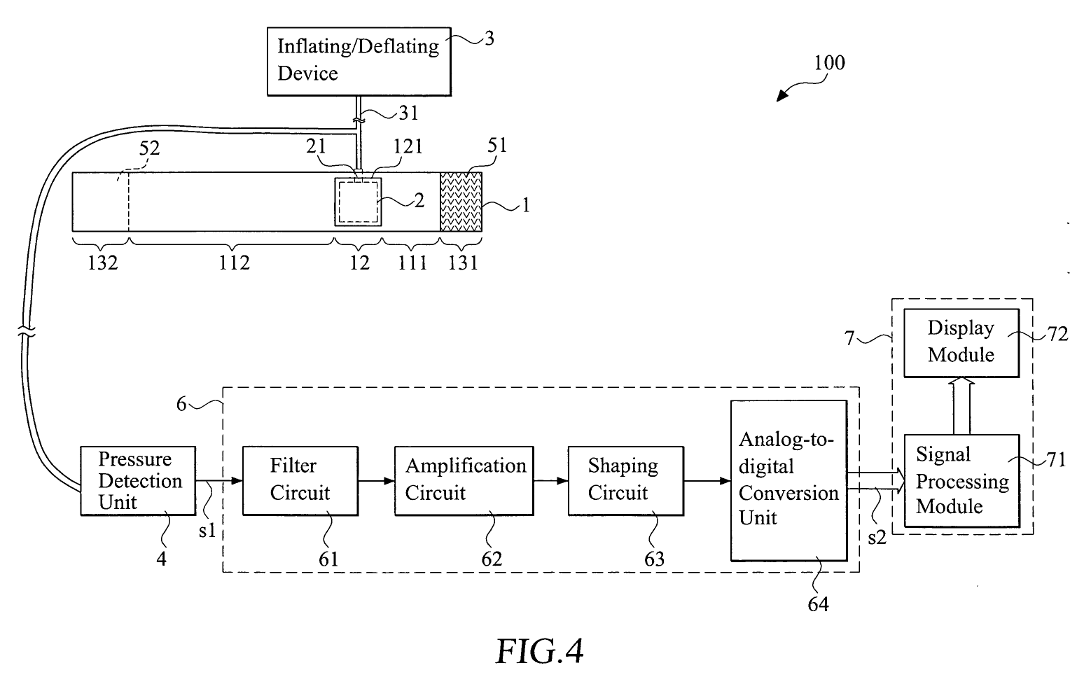

With reference to the drawings and in particular to FIGS. 1-4, a carotid pulse measurement device constructed in accordance with the present invention, generally designated with reference numeral 100, comprises a flexible band 1, an artery-pressing air cell 2, an inflating/deflating device 3, and a pressure detection unit 4. The band 1, serving to surround a user's neck, comprises two enclosing sections 111, 112 and an effective pressing section 12. The band 1 also has two ends forming fastening sections 131, 132 respectively. Fastener elements 51, 52 are mounted to the fastening sections 131, 132 respectively.

To wear the band 1 around the neck, the enclosing sections 111, 112 are disposed to surround the neck with the effective pressing section 12 positioned corresponding to the carotid artery of the user's neck. The band 1 is then fixed around the user's neck by the fastener elements 51, 52 and adjustability can be done in accordance with the size of the user's neck. The band 1 has a skin-contact surface 10, formed by one of opposite cover layers of the band 1, on which an air-cell location indication zone 121 is formed at a location corresponding to the effective pressing section 12.

The air cell 2 comprises a nozzle 21 and a plurality of inflatable chambers 22, 23, 24. The chambers 22, 23, 24 are in fluid communication with each other and the nozzle 21, and are arranged in a stacked manner between the two cover layers of the band 1 and thus inside the band 1. The air cell 2 is located corresponding to the effective pressing section 12 whereby the user may readily get aware of the location of the air cell 2 by means of the air-cell location indication zone 121. The inflating/deflating device 3 is connected by an inflation tube 31 to the nozzle 21 of the air cell 2 so that the inflating/deflating device 3 can selectively inflate/deflate the air cell 2.

The pressure detection unit 4 is connected by the inflation tube 31 of the inflating/deflating device 3 to the air cell 2 for detection of pressure variation inside the air cell 2.

To use the measurement device 100, the band 1 is put around the user's neck and the inflating/deflating device 3 fills air into the air cell 2. The pressure detection unit 4 is then operated to detect the pressure variation inside the inflation tube 31 of the inflating/deflating device 3 and the air cell 2 and, in response to the detection, generates a pulse signal s1, which is then applied to a pulse signal processing module 6.

The pulse signal processing module 6 comprises a filter circuit 61, an amplification circuit 62, a shaping circuit 63, and an analog-to-digital conversion unit 64. The pulse signal s1 from the pressure detection unit 4 is sequentially applied through the filter circuit 61, the amplification circuit 62, and the shaping circuit 63 for noise filtration and pre-amplification and shaping of the signal. The so-processed signal is then fed through the analog-to-digital conversion unit 64 for conversion of the pulse signal s1 into a digital signal s2.

The pulse signal processing module 6 is connected to a computer device 7. The computer device 7 comprises a signal processing module 71 and a display module 72. The signal processing module 71 receives the digital signal s2 from the pulse signal processing module 6 and the signal, after processed by the signal processing module 71, is displayed on the display module 72.

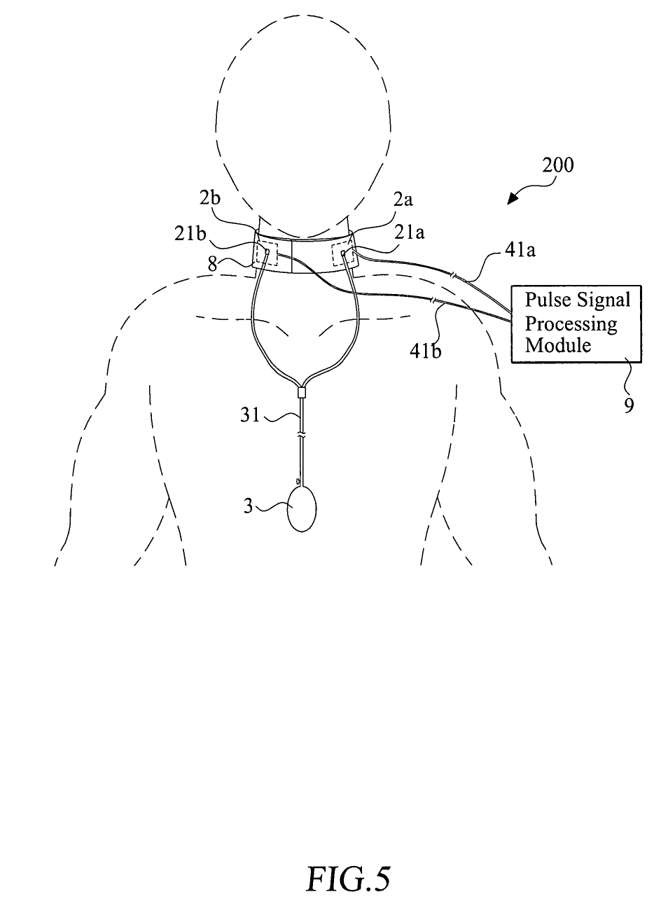

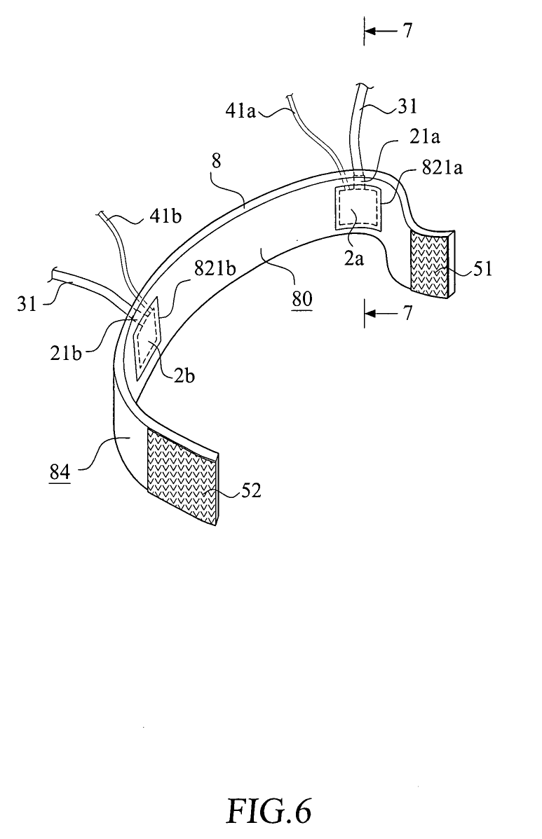

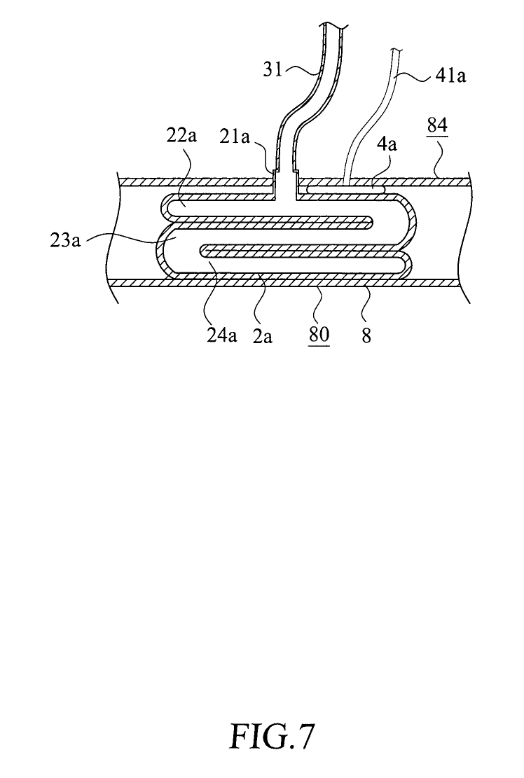

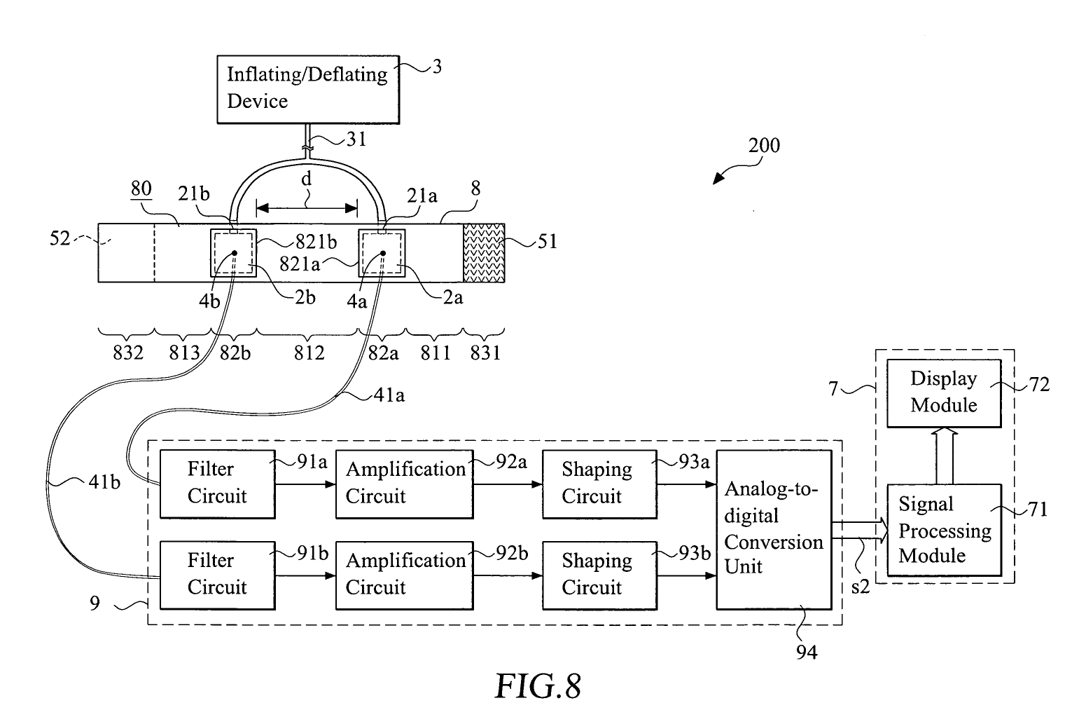

Referring now to FIGS. 5-8, wherein a second embodiment of the carotid pulse measurement device in accordance with the present invention, generally designated with reference numeral 200, is shown, the carotid pulse measurement device 200 comprises a band 8, a first artery-pressing air cell 2a, a second artery-pressing air cell 2b, an inflating/deflating device 3, a first pressure detection unit 4a, and a second pressure detection unit 4b. The band 8, serving to surround a user's neck, comprises enclosing sections 811, 812, 813, and a first effective pressing section 82a and a second effective pressing section 82b alternating the enclosing sections 811, 812, 813. The first and second effective pressing sections 82a, 82b are spaced from each other by a predetermined separation distance d. The band 8 has two ends respectively forming fastening sections 831, 332 to which the fastener elements 51, 52 are mounted.

To wear the band 8 around the neck, the enclosing sections 811, 812, 813 are put around the neck with the first and second effective pressing sections 82a, 82b respectively corresponding in position to the left and right carotid arteries of the user's neck. The band 8 is then fixed around the user's neck by the fastener elements 51, 52 in an adjustable manner in accordance with the size of the user's neck. The band 8 has a skin-contact surface 80 on which first and second air-cell location indication zones 821a, 821b are formed at locations corresponding to the first and second effective pressing sections 82a, 82b respectively.

The first air cell 2a comprises a nozzle 21a and a plurality of inflatable chambers 22a, 23a, 24a, which are in fluid communication with each other and the nozzle 21a and are arranged in a stacked manner inside the band 8. The first air cell 2a is located corresponding to the first effective pressing section 82a, namely comprising a nozzle 21b and chambers (not shown) in fluid communication with each other and stacked inside the band 8. The second air cell 2b is of a construction identical to the first air cell 2a and is located corresponding to the second effective pressing section 82b.

The user may readily get aware of the locations of the first and second air cells 2a, 2b by means of the first and second air-cell location indication zones 821a, 821b respectively. The inflating/deflating device 3 is connected by an inflation tube 31 to the nozzles 21a, 21b of the first and second air cells 2a, 2b so that the inflating/deflating device 3 can selectively inflate/deflate the air cells 2a, 2b.

The first pressure detection unit 4a is arranged between an air-contact surface 84 of the band 8 and the first air cell 2a to allow the first pressure detection unit 4a to detect pressure variation of the first air cell 2a. The second pressure detection unit 4b is arranged between the air-contact surface 84 of the band 8 and the second air cell 2b to allow the second pressure detection unit 4b to detect pressure variation of the second air cell 2b.

To use the measurement device 200, the band 8 is put around the user's neck and the inflating/deflating device 3 fills air into the first and second air cells 2a, 2b. The first pressure detection unit 4a detects the pressure variation of the first air cell 2a due to a reaction force induced by the pulse of the carotid artery of one side of the user's neck, for example, the left side carotid artery, and, in response to the detection, generates a first pulse signal (not explicitly shown in the drawings). The second pressure detection unit 4b is operated in the same way as the first pressure detection unit 4a, but is used to detect the pressure variation of the second air cell 2b and generating a second pulse signal in response to the detection. The first and second pulse signals are then applied to a pulse signal processing module 9 via first and second signal transmission lines 41a, 41b respectively.

The pulse signal processing module 9 comprises two filter circuits 91a, 91b, two amplification circuits 92a, 92b, two shaping circuits 93a, 93b, and an analog-to-digital conversion unit 94. The first pulse signal from the first pressure detection unit 4a and the second pulse signal from the second pressure detection unit 4b are respectively and sequentially applied through the filter circuits 91a, 91bb, the amplification circuits 92a, 92b, and the shaping circuits 93a, 93b for noise filtration and pre-amplification and shaping of the signals. The so-processed signals are then fed through the analog-to-digital conversion unit 94 for conversion of the first and second pulse signals into a digital signal s2.

The pulse signal processing module 9 is connected to a computer device 7. The computer device 7 comprises a signal processing module 71 and a display module 72. The signal processing module 71 receives the digital signal s2 from the pulse signal processing module 9 and the signal, after processed by the signal processing module 71, is displayed on the display module 72.

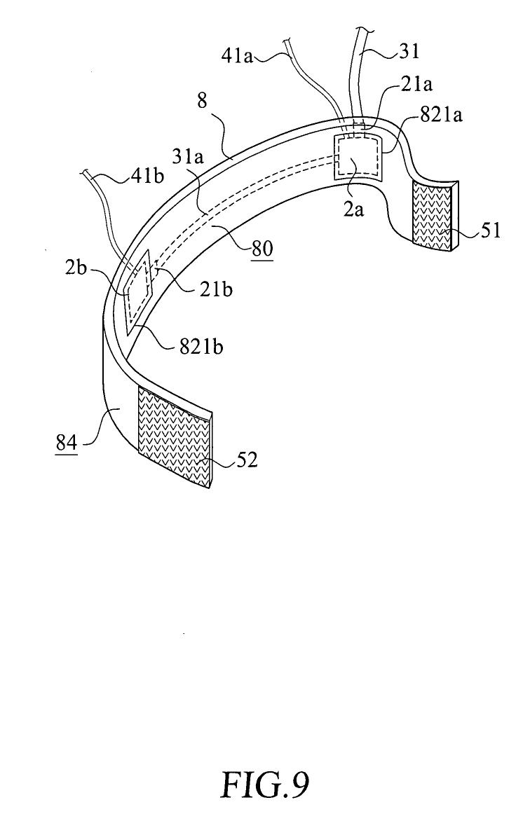

Referring to FIG. 9, which shows a band that is used in a carotid pulse measurement device constructed in accordance with a third embodiment of the present invention, which is a modification of the second embodiment discussed with reference to FIGS. 5-8 previously, although being indicated with the same reference numeral 8, the band of the third embodiment is different from the band of the second embodiment in that the nozzle 2b of the second air cell 2b is fluidly connected to the first air cell 2a by an inter-cell inflation tube 31a so that the second air cell 2b is inflated by the inter-cell inflation tube 31a only at the time when the inflating/deflating device 3 is inflating the first air cell 2a.

In accordance with the present invention, the chambers 22, 23, 24 of the air cell 2, or the chambers 22a, 23a, 24a of the first air cell 2a or the chambers 22b, 23b, 24b of the second air cell 2b can be folded in different ways and with different number of folds according practical requirements and uses of the measurement devices 100, 200. In a practical embodiment, the air cell 2 and first and second air cells 2a, 2b have a square shape having an area of 16 cm2. The size and shape of the air cells 2, 2a, 2b may also be changed according practical requirements and uses of the measurement devices 100, 200.

To this point, it is understood that the in the present invention, the air cell 2 or first and second air cells 2a, 2b are arranged inside the band 1 or 8 at positions of the carotid arteries of the user's neck for efficient measurement of the artery pulse in a way that does not cause substantial pressure to the user's neck and thus does not cause discomfort and strain. In addition, according to the second and third embodiments, the present invention is also advantageous in employing the first and second air cells 2a, 2b respectively on the left and right side carotid arteries. This allows the left and right carotid pulse to be measured at the same time by the first and second pressure detection units 4a, 4b.

When the band 8 is worn around the user's neck, if the air cells are not positioned exactly corresponding to the carotid arteries, the pulse signal processing module 9 may select and use the stronger one of the first and second pulse signals generated by the first and second pressure detection units 4a, 4b to generate the digital pulse signal s2. This is helpful in reducing human error in securing the band to the user's neck.

In addition, the user may use the first pulse signal from the first pressure detection unit 4a and/or the second pulse signal from the second pressure detection unit 4b, together with pulse waveform obtained from peripheral vessels or extremity vessels (such as vessels of fingers or toes), to calculate arteriosclerosis index to evaluate the degree of arteriosclerosis.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A carotid pulse measurement device adapted to measure pulse of carotid artery of a user, comprising:
a band adapted to surround a neck of the user, the band comprising an enclosing section and an effective pressing section;
an air cell arranged in the band at a location corresponding to the effective pressing section, and comprising a nozzle;
an inflating/deflating device fluidly connected to the nozzle of the air cell via an inflation tube for selectively inflating/deflating the air cell; and
a pressure detection unit arranged to detect pressure variation of the air cell and, in response thereto, to generate a pulse signal;
wherein to use the measurement device, the enclosing section of the band is put around the neck of the user to position the effective pressing section at a location corresponding to the carotid artery of the user's neck.

2. The carotid pulse measurement device as claimed in claim 1, wherein the air cell comprises a plurality of inflation chambers, which are arranged inside the band in a stacked manner and are in fluid communication with each other and the nozzle.

3. The carotid pulse measurement device as claimed in claim 1, wherein the band has a skin-contact surface adapted to oppose skin of the user and forming an air-cell location indication zone at a location corresponding to the effective pressing section to allow ready recognition of the location of the air cell.

4. The carotid pulse measurement device as claimed in claim 1, wherein the pressure detection unit is fluidly connected to the air cell via the inflation tube of the inflating/deflating device so as to allow detection of pressure of the air cell and the inflation tube by the pressure detection unit.

5. The carotid pulse measurement device as claimed in claim 1, wherein the pressure detection unit is arranged between an air-contact surface of the band and the air cell to allow the pressure detection unit to detect the pressure variation of the air cell by means of reaction induced by the carotid pulse.

6. The carotid pulse measurement device as claimed in claim 1, wherein the band has two ends forming fastening sections on which fastener elements are mounted respectively for releasably securing the band around the user's neck.

7. A carotid pulse measurement device adapted to measure pulse of carotid artery of a user, comprising:
a band adapted to surround a neck of the user, the band comprising an enclosing section, a first effective pressing section, and a second effective pressing section, the first and second effective pressing sections being spaced by a distance;
a first air cell arranged in the band at a location corresponding to the first effective pressing section, and comprising a nozzle;
a second air cell arranged in the band at a location corresponding to the second effective pressing section, and comprising a nozzle;
an inflating/deflating device operable to selectively inflate/deflate the first and second air cells;
a first pressure detection unit operable to detect pressure variation of the first air cell and, in response thereto, to generate a first pulse signal; and
a second pressure detection unit operable to detect pressure variation of the second air cell and, in response thereto, to generate a second pulse signal;
wherein to use the measurement device, the enclosing section of the band is put around the neck of the user to position the first and second effective pressing section at locations corresponding to left and right carotid arteries respectively.

8. The carotid pulse measurement device as claimed in claim 7, wherein the inflating/deflating device is fluidly connected to the nozzle of the first air cell via an inflation tube for selectively inflating/deflating the first air cell.

9. The carotid pulse measurement device as claimed in claim 7, wherein the nozzle of the second air cell is fluidly connected to the first air cell via an inter-cell inflation tube so that when the inflating/deflating device is operated to inflate/deflate the first air cell, the second air cell is selectively inflated/deflated via the inter-cell inflation tube.

10. The carotid pulse measurement device as claimed in claim 7, wherein the inflating/deflating device is fluidly connected to the nozzle of the second air cell via an inflation tube for selectively inflating/deflating the second air cell.

11. The carotid pulse measurement device as claimed in claim 7, wherein the first air cell comprises a plurality of inflation chambers, which are arranged inside the band in a stacked manner and are in fluid communication with each other.

12. The carotid pulse measurement device as claimed in claim 7, wherein the second air cell comprises a plurality of inflation chambers, which are arranged inside the band in a stacked manner and are in fluid communication with each other.

13. The carotid pulse measurement device as claimed in claim 7, wherein the band has a skin-contact surface adapted to oppose skin of the user and forming a first air-cell location indication zone at a location corresponding to the first effective pressing section to allow ready recognition of the location of the first air cell.

14. The carotid pulse measurement device as claimed in claim 7, wherein the band has a skin-contact surface adapted to oppose skin of the user and forming a second air-cell location indication zone at a location corresponding to the second effective pressing section to allow ready recognition of the location of the second air cell.

15. The carotid pulse measurement device as claimed in claim 7, wherein the first pressure detection unit is fluidly connected to the first air cell via an inflation tube of the inflating/deflating device so as to allow detection of pressure of the first air cell and the inflation tube by the first pressure detection unit.

16. The carotid pulse measurement device as claimed in claim 7, wherein the second pressure detection unit is fluidly connected to the second air cell via an inflation tube of the inflating/deflating device so as to allow detection of pressure of the second air cell and the inflation tube by the second pressure detection unit.

17. The carotid pulse measurement device as claimed in claim 7, wherein the first pressure detection unit is arranged between an air-contact surface of the band and the first air cell to allow the first pressure detection unit to detect the pressure variation of the first air cell by means of reaction induced by the carotid pulse.

18. The carotid pulse measurement device as claimed in claim 7, wherein the second pressure detection unit is arranged between an air-contact surface of the band and the second air cell to allow the second pressure detection unit to detect the pressure variation of the second air cell by means of reaction induced by the carotid pulse.

19. The carotid pulse measurement device as claimed in claim 7, wherein the band has two ends forming fastening sections on which fastener elements are mounted respectively for releasably securing the band around the user's neck.

20. An artery-pressing air cell adapted to be arranged inside a flexible band functioning to surround a user's neck, the air cell comprising a nozzle for inflating the air cell, the air cell comprising a plurality of inflation chambers that are arranged in a folded and stacked manner and are in fluid communication with each other and the nozzle.
